(19) United States
(12) Patent Application Publication
(10) Pub. No.: US 2006/0116558 A1
(43) Pub. Date: Jun. 1, 2006

(54) Health care method and apparatus based on user's life pattern

(76) Inventors: Woo-Young Jang, Seoul (KR); Yeun-bee Kim, Seongnam-si (KR); Kwang-hyeon Lee, Yongin-si (KR); Jin-sang Hwang, Suwon-si (KR)

Correspondence Address: STAAS & HALSEY LLP, SUITE 700, 1201 NEW YORK AVENUE, N.W., WASHINGTON, DC, 20005, US

(21) Appl. No.: 11/271,967

(22) Filed: Nov. 14, 2005

(30) Foreign Application Priority Data
Nov. 11, 2004 (KR) 10-2004-0091848

(51) Int. Cl. A61B 5/00

(52) U.S. Cl. 600/300; 128/920; 128/921

(57) ABSTRACT

A health care apparatus and method that produce a health care program based on a user's life pattern. The health care apparatus includes: an input unit inputting user's personal information, body information, and life pattern information; a calculator producing a health care program based on the input user's personal information, body information, and life pattern information; and an output unit outputting the produced health care program. The health care method and apparatus produce a health care program based on a user's life pattern that reflects user's nutritive food intake and user's consumed nutritive food, thereby providing a user with a more accurate and reliable health care program.

### CROSS-REFERENCE TO RELATED APPLICATION

This application claims the benefit of Korean Patent Application No. 10-2004-0091848, filed on Nov. 11, 2004, in the Korean Intellectual Property Office, the disclosure of which is incorporated herein by reference.

### BACKGROUND OF THE INVENTION

1. Field of the Invention

The present invention relates to a health care method and system, and more particularly, to a health care method and apparatus for producing a health care management program based on a user's life pattern and providing additional information for a user's predetermined goal to promote the attainment of the user's goal.

2. Description of Related Art

Recently, the demand for a health care method and apparatus that provide a health care program adapted to users has increased as people pay ever increasing attention to their health.

Korean Patent Publication No. 10-2002-0006980 discloses a system and method for providing health information based on a diet diary obtained from a plurality of pieces of diet information most suitable for a user using user's body information.

Korean Patent Publication No. 10-2002-0010293 discloses a computer network-based total health care system and method that extract user's health information through a computer network and provide the extracted health information at the request of a user.

These conventional technologies simply receive user's body information and provide health information corresponding to the received user's body information. Therefore, they do not provide a health care program based on a user's scheduled life pattern. Also, they simply provide the user with the health care program produced based on the received user's body information. Therefore, they do not actively provide the health care program according to the user's scheduled life pattern.

### BRIEF SUMMARY

An aspect of the present invention provides a health care apparatus for producing a health care program based on a user's life pattern.

An aspect of the present invention also provides a health care method for producing a health care program based on a user's life pattern.

According to an aspect of the present invention, there is provided a health care apparatus including: an input unit inputting a user's personal information, body information, and life pattern information; a calculator producing a health care program based on the input user's personal information, body information, and life pattern information; and an output unit outputting the produced health care program.

According to another aspect of the present invention, there is provided a health care method including: inputting a user's personal information, body information, and life pattern information; producing a user's life pattern based on the user's life pattern information; producing the health care program based on the user's personal information, body information, and life pattern information; and outputting the produced health care program to the user.

According to still another aspect of the present invention, there is provided a computer-readable storage medium encoded with processing instructions for causing a processor to execute the aforesaid method.

Additional and/or other aspects and advantages of the present invention will be set forth in part in the description which follows and, in part, will be obvious from the description, or may be learned by practice of the invention.

### BRIEF DESCRIPTION OF THE DRAWINGS

The above and/or other aspects and advantages of the present invention will become apparent and more readily appreciated from the following detailed description, taken in conjunction with the accompanying drawings of which:

FIG. 1 is a block diagram of a health care apparatus for producing a health care program based on a user's life pattern according to an embodiment of the present invention;

FIG. 2 is a block diagram of a calculator shown in FIG. 1;

FIG. 3 is a block diagram of a life pattern production agent shown in FIG. 2;

FIG. 4, parts (a) through (d), are tables that categorize an amount of user's nutritive food intake and an amount of user's consumed nutritive food based on users' food and sport information;

FIGS. 5 through 7 illustrate a user's life schedule, a calorie intake/consumption pattern, and a sugar intake/consumption pattern corresponding to the user's life schedule;

FIG. 8 is a block diagram of a health care program production agent shown in FIG. 2;

FIG. 9 is a block diagram of an additional information production agent shown in FIG. 2;

FIG. 10 is a flowchart of a health care method based on a user's life pattern according to an embodiment of the present invention;

FIG. 11 is a flowchart of Operation 1020 shown in FIG. 10; and

FIG. 12 is a flowchart of Operation 1030 shown in FIG. 10.

### DETAILED DESCRIPTION OF EMBODIMENTS

Reference will now be made in detail to embodiments of the present invention, examples of which are illustrated in the accompanying drawings, wherein like reference numerals refer to the like elements throughout. The embodiments are described below in order to explain the present invention by referring to the figures.

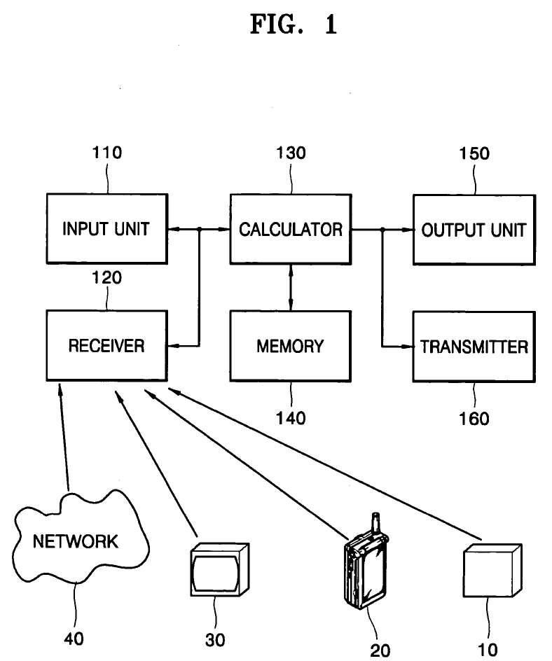

FIG. 1 is a block diagram of a health care apparatus that produces a health care program based on a user's life pattern according to an embodiment of the present invention. Referring to FIG. 1, the health care apparatus includes an input unit 110, a receiver 120, a calculator 130, a memory 140, an output unit 150, and a transmitter 160.

The input unit 110 inputs user's personal information, body information, and life pattern information. The input unit 110 may be a mouse, a keyboard, a touch screen, etc. for inputting the user's personal information, body information, and life pattern information.

The receiver 120 receives the user's personal information, body information, and life pattern information from a mobile device, e.g., a sensor 10, a cellular phone 20, a personal digital assistant (PDA) 30, or a network 40 connected to the health care apparatus.

The sensor 10 senses a user's sport using an acceleration sensor and a terrestrial magnetic sensor and determines a type of a user's action based on the sensed sport. For example, the sensor 10 determines whether a user is taking a seat, walking, running, or going up stairs. The user inputs his personal information, body information, and life pattern information using the cellular phone 20 or the PDA 30. The receiver 120 receives the user's personal information, body information, and life pattern information from the cellular phone 20 or the PDA 30.

The health care apparatus is connected to the network 40, and the receiver 120 is connected to the network 40 at the request of the calculator 130 to receive the user's personal information, body information, and life pattern information.

The user's personal information may include user's name, ID number, family particulars, age, marriage status, disease history, goal, favorite food, and favorite sport. The user's body information may include user's weight, height, blood pressure, fat, sugar value, blood components. The user's life pattern information includes food information on kinds and amounts of a user's food intake and sport information on kinds and time of sport performed by the user.

The calculator 130 classifies the information obtained from the input unit 110 or the receiver 120 into the user's personal information, body information, and life pattern information, stores the classified information in the memory 140, and produces a health care program most suitable for the user based on the stored information.

The produced health care program is output though the output unit 150 such as a display (not shown) or a printer (not shown), or transmitted to an external device (not shown) such as a cellular phone (not shown) or a PDA (not shown) through the transmitter 160. The user changes an amount of user's nutritive food intake and an amount of user's consumed nutritive food based on the output or transmitted health care program, thereby providing a health care program adaptive to the user.

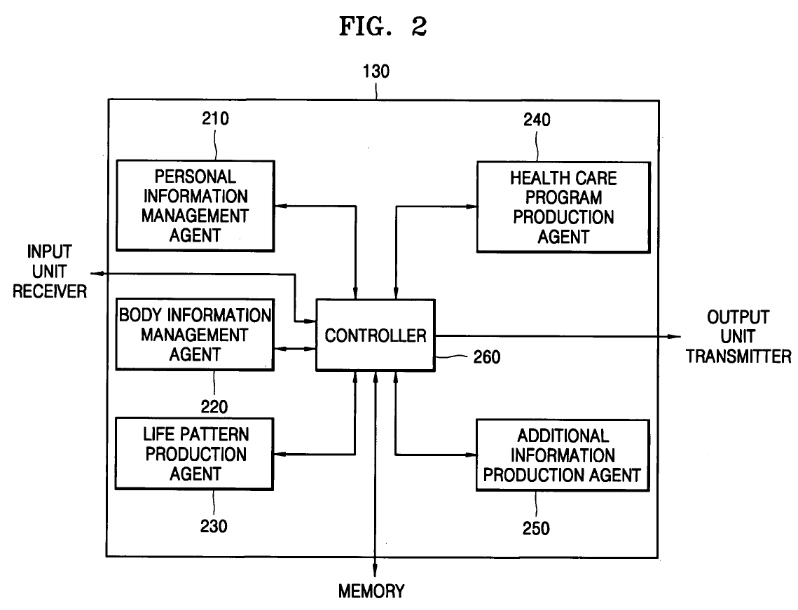

FIG. 2 is a block diagram of the calculator 130 shown in FIG. 1. Referring to FIG. 2, the calculator 130 includes a personal information management agent 210, a body information management agent 220, a life pattern production agent 230, a health care program production agent 240, an additional information production agent 250, and a controller 260.

Referring to FIGS. 1 and 2, the personal information management agent 210 extracts the user's personal information through the input unit 110 or the receiver 120, registers the extracted user's personal information in the memory 140, and if another agent requests the extracted user's personal information, extracts the user's personal information from the memory 140 through the controller 260, and provides the other agent with the extracted user's personal information.

The body information management agent 220 extracts the user's body information through the input unit 110 or the receiver 120, registers the extracted user's body information in the memory 140, and if other agent requests the extracted user's body information, extracts the user's body information from the memory 140 through the controller 260, and provides the other agent with the extracted user's body information. The body information management agent 220 regularly searches for the extracted user's body information, and if the searched user's body information reveals something wrong, it outputs the wrong user's body information to the user using the output unit 150 through the controller 260 or to a user's external device using the transmitter 160.

The life pattern production agent 230 extracts the user's life pattern information, i.e., user's food and sport information, using the input unit 110 or the receiver 120. The user's food information includes information on a kind and an amount of user's food intake and the user's sport information includes information on a kind and time of sport taken by the user. The user's food and sport information is input by the user or automatically by the sensor 10, the cellular phone 20, the PDA 30, and the network 40. The life pattern production agent 230 can extract the user's sport information using an on/off operating signal transmitted from household appliances, e.g., a television, a refrigerator, a computer, etc., or from illuminators.

The life pattern production agent 230 maps the amount of user's food intake to a predetermined category of nutritive food ingestion based on the kind and amount of user's food intake of the extracted food information to calculate the amount of user's food intake. The life pattern production agent 230 maps the amount of user's consumed food to a predetermined category of nutritive food ingestion based on the kind and time of sport taken by the user of the extracted sport information to calculate the amount of user's consumed food. The life pattern production agent 230 produces a user's life pattern based on the calculated amount of user's food intake and amount of user's consumed food. The life pattern production agent 230 registers the produced user's life pattern to the memory 140, and if other agent requests the produced user's life pattern, extracts the user's life pattern from the memory 140 through the controller 260, and provides the other agent with the extracted user's life pattern.

The health care program production agent 240 produces a health care program adapted to the user based on the user's life pattern produced from the life pattern production agent 230. The health care program production agent 240 calculates a target amount of nutritive food intake and a target consumption amount of nutritive food based on the user's personal information and body information, reflects the produced user's life pattern on the calculated target amount of nutritive food intake and target consumption amount of nutritive food, and calculates a modified target amount of nutritive food intake and target consumption amount of nutritive food. The health care program production agent 240 produces the health care program adapted to the user based on the modified target amount of nutritive food intake and target consumption amount of nutritive food.

The additional information production agent 250 extracts additional information relating to the user's personal information and body information containing a user's predetermined target through a network connected to the receiver 120 and outputs the extracted additional information using the output unit 150 such as a display or a printer, or transmits the extracted additional information to the user's external device through the transmitter 160. The additional information may be, for example, results of a medical examination of people related to the user's personal information or the user's body information, media information related to the user's personal information or the user's body information, community information related to the user's personal information or the user's body information, and the like.

The controller 260 manages information input or output to each of the agents 210 through 250. The controller 260, if one of the agents 210 through 250 requests predetermined information, extracts the predetermined information from an agent managing the predetermined information and provides the agent requesting the predetermined information with the extracted information. The controller 260 extracts predetermined information using the receiver 120 at the request of each of the agents 210 through 250 and provides each of the agents 210 through 250 with the extracted information, or outputs the predetermined information using the output unit 150 at the request of each of the agents 210 through 250 or transmits the extracted information to each of the agents 210 through 250 through the transmitter 160.

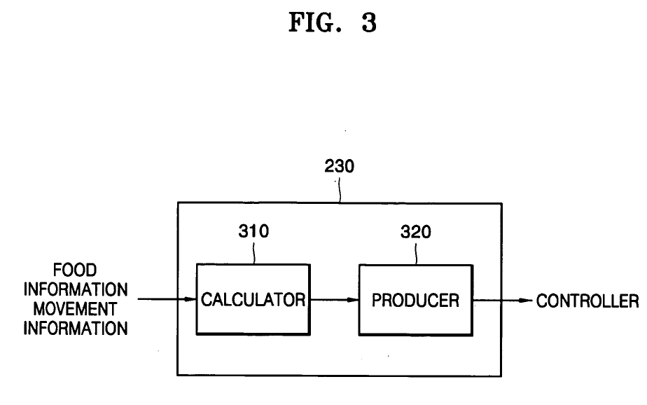

FIG. 3 is a block diagram of the life pattern production agent 230 shown in FIG. 2. Referring to FIGS. 1-3, the life pattern production agent 230 extracts user's life pattern information from the input unit 110 or the receiver 120 through the controller 260 and produces a user's life pattern based on the extracted user's life pattern information. A calculator 310 of the life pattern production agent 230 receives the user's life pattern information, i.e., user's food and sport information, and calculates an amount of user's nutritive food intake and an amount of user's consumed nutritive food. The calculator 310 preferably calculates the amount of user's nutritive food intake and the amount of user's consumed nutritive food based on a table that categorizes an amount of nutritive food intake according to the kind and amount of food stored in the memory 140 and a table that categorizes a consumption amount of nutritive food according to the kind and time of sport stored in the memory 140. A producer 320 of the life pattern production agent 230 produces the user's life pattern based on the calculated amount of user's nutritive food intake and amount of user's consumed nutritive food.

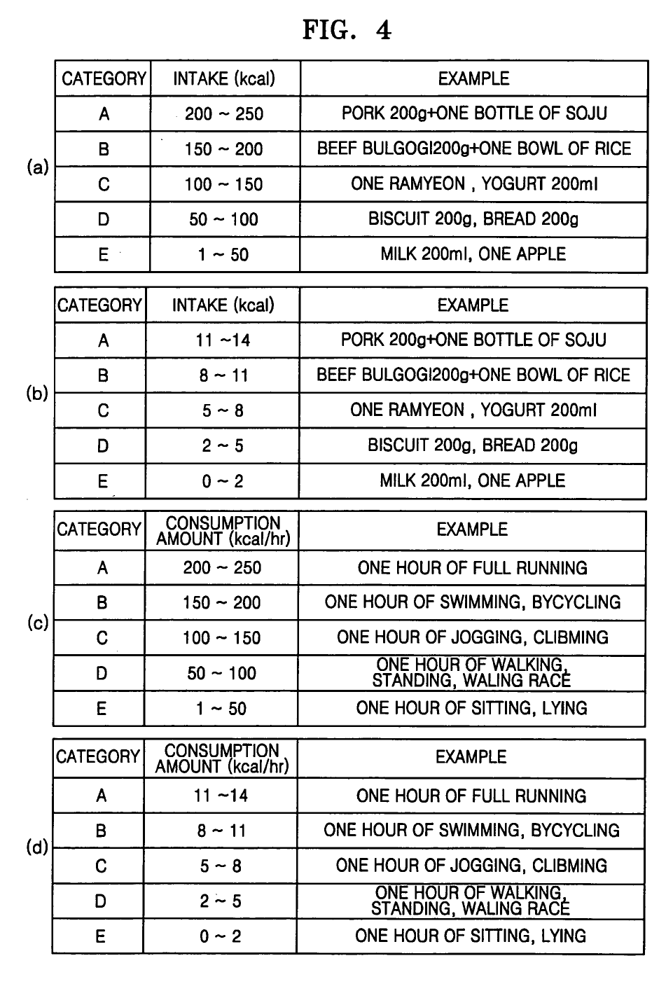

FIG. 4, parts (a) through (d) are tables that categorize an amount of user's nutritive food intake and an amount of user's consumed nutritive food based on users' food and sport information. The tables exemplarily categorize an amount of user's nutritive food intake according to kinds and amount of food and an amount of user's consumed nutritive food according to kinds of time of sport and can categorize other kinds of nutritive food in the same manner.

Part (a) of FIG. 4 shows a table categorizing amounts of user's caloric intake according to kinds and amount of user's food intake. Referring to part (a) of FIG. 4, amounts of the user's caloric intake are categorized according to the kinds and amount of user's food intake and are calculated according to kinds and amount of similar or identical food based on the categorized amounts of the user's caloric intake.

Part (b) of FIG. 4 shows a table categorizing a user's carbohydrate intake according to kinds and amount of user's food intake. Referring to part (b) of FIG. 4, amounts of the user's carbohydrate intake are categorized according to the kinds and amount of user's food intake and are calculated according to kinds and amount of similar or identical food based on the categorized amounts of the user's carbohydrate intake.

Part (c) of FIG. 4 shows a table categorizing a user's caloric expenditure according to kinds and time of a user's sport. Referring to part (c) of FIG. 4, amounts of the user's consumed calorie are categorized according to the kinds and time of a user's sport are calculated according to kinds and time of similar or identical sport based on the categorized amounts of the user's consumed calorie.

Part (d) of FIG. 4 shows a table categorizing a user's consumed sugar according to kinds and time of a user's sport. Referring to part (d) of FIG. 4, amounts of the user's consumed sugar are categorized according to the kinds and time of the user's sport and are calculated according to kinds and time of a similar or identical sport based on the categorized amounts of the user's consumed sugar.

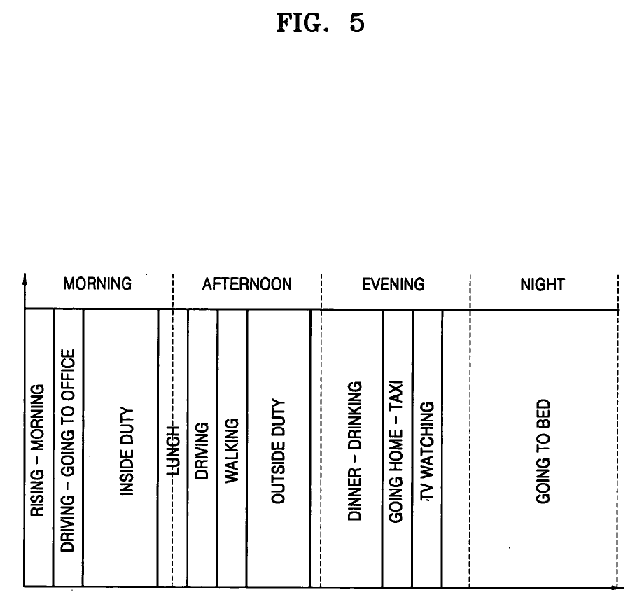

FIGS. 5 through 7 illustrate a user's life schedule and a calorie intake/consumption pattern and a sugar intake/consumption pattern corresponding to the user's life schedule.

FIG. 5 exemplarily illustrates a user's day schedule to produce the user's life pattern. Referring to FIGS. 1 and 5, the user's day schedule includes sport information on duties performed by the user, TV watching, etc., and food information. The user's day schedule is directly input by the user through the input unit 110 or automatically input by receiving the user's life pattern information transmitted through the sensor 10, the cellular phone 20, the PDA 30, the network 40, etc., used by the user using the receiver 120.

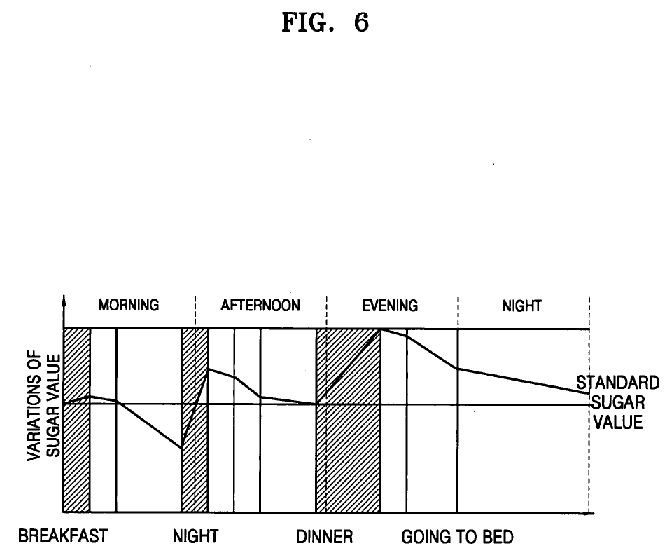

FIG. 6 is a graph of sugar intake and consumption variations according to the user's day schedule. Referring to FIG. 6, a sugar value is very low since the user had a small breakfast and rapidly increases since the user had pork and alcohol at dinner. Therefore, it can be informed from the user's sugar intake and consumption that the user had a smaller sugar intake than a target sugar intake at breakfast and a more sugar intake than the target sugar intake at dinner.

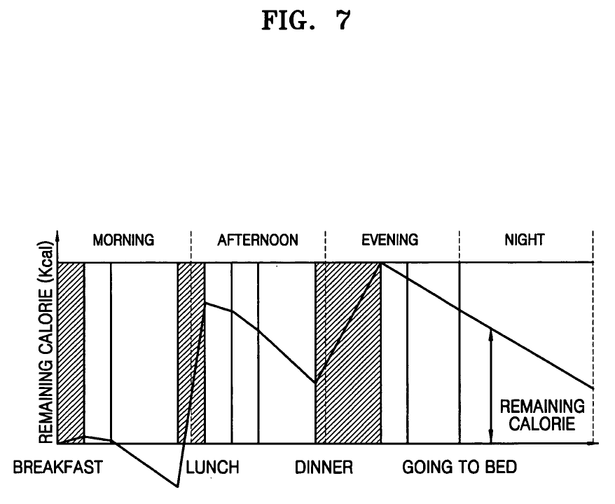

FIG. 7 is a graph of caloric intake and consumption variations according to the user's day schedule. Referring to FIG. 7, the user had a small breakfast and is short of a caloric intake, had a lot of caloric intake at lunch due to hunger, had pork and alcohol at dinner and additionally had calorie. Therefore, the graph show that the user had much more calorie intake than the target caloric intake and a lot of calorie still remains.

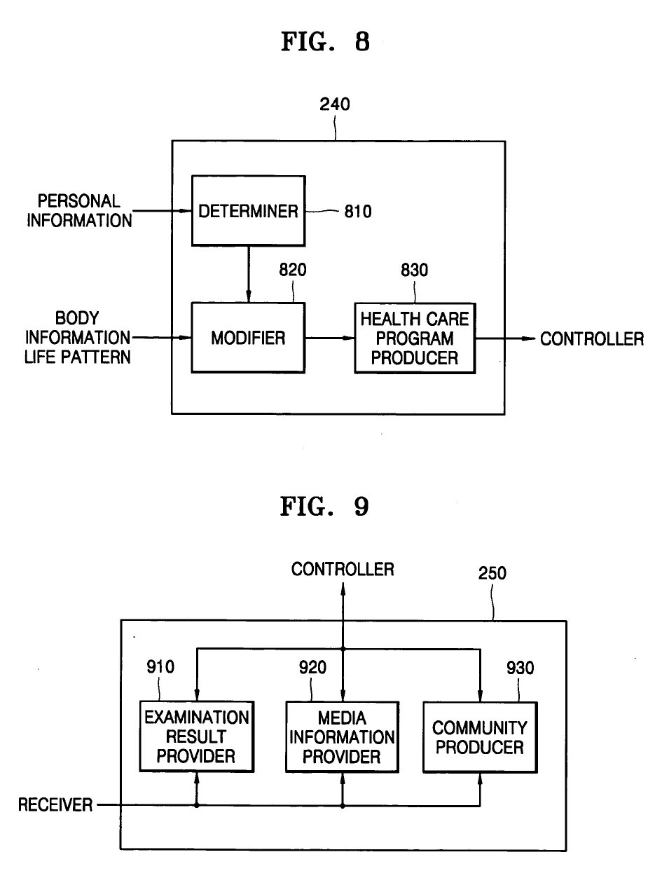

FIG. 8 is a block diagram of the health care program production agent 240 shown in FIG. 2. Referring to FIGS. 1, 2, and 8, the health care program production agent 240 includes a determiner 810, a modifier 820, and a health care program producer 830.

The determiner 810 extracts user's personal information and body information from the input unit 110 or the receiver 120 through the controller 260 and determines a target amount of nutritive food intake and a target consumption amount of nutritive food based on the extracted user's personal information and body information. The determiner 810 determines the target amount of nutritive food intake and the target consumption amount of nutritive food that satisfy a user's input predetermined target. For example, if the user wants to lose 3 kg in a month, the determiner 810 determines the target amount of nutritive food intake and the target consumption amount of nutritive food for the 3 kg loss in weight for a month based on the extracted user's personal information and body information. Also, if the user's predetermined target is not established, the determiner 810 determines a standard amount of nutritive food intake and a standard consumption amount of nutritive food as the target amount of nutritive food intake and the target consumption amount of nutritive food based on the extracted user's personal information and body information, e.g., user's age, height, weight, etc. The standard amount of nutritive food intake and the standard consumption amount of nutritive food are to maintain a standard body condition based on a standard weight, a standard fat, etc., corresponding to the user's age, height, etc.

The modifier 820 modifies the target amount of nutritive food intake and the target consumption amount of nutritive food based on the user's life pattern produced from the life pattern production agent 230. In detail, if the user has a more nutritive food intake than the target amount of nutritive food intake or the user has a smaller amount of nutritive food consumption that the target consumption amount of nutritive food, the modifier 820 modifies the target amount of nutritive food intake and the target consumption amount of nutritive food. The health care program producer 830 produces a user's health care program based on the modified amount of nutritive food intake and consumption amount of nutritive food. For example, when the user has a more nutritive food intake than the target amount of nutritive food intake at dinner, the health care program producer 830 produces a user's health care program, e.g., walking home from work, or riding a bicycle for an hour before retiring, to accomplish the modified consumption amount of nutritive food. The produced health care program is output to the user through the output unit 150 or transmitted to a user's external device through the transmitter 160.

FIG. 9 is a block diagram of the additional information production agent 250 shown in FIG. 2. Referring to FIGS. 1, 2, and 9, the additional information production agent 250 produces additional information relating to the user's personal information and body information. The additional information production agent 250 includes an examination result provider 910, a media information provider 920, and a community producer 930.

The examination result provider 910 receives results of medical examinations of people having similar information to the user's personal information and body information using the receiver 120 through a predetermined network and provides the user with the received results.

The media information provider 920 receives information on media relating to the user's personal information and body information using the receiver 120 through the predetermined network and provides the user with the received media information.

The community producer 930 searches for a community relating to the user's personal information and body information through the predetermined network and provides the user with the searched community, or automatically produces the community relating to the user's personal information and body information and provides the user with the produced community. The user can obtain information relating to the user's personal information and body information from the additional information and more actively cares for his health.

The additional information production agent 250 can produce different additional information and provide the produced additional information within the scope of the present invention.

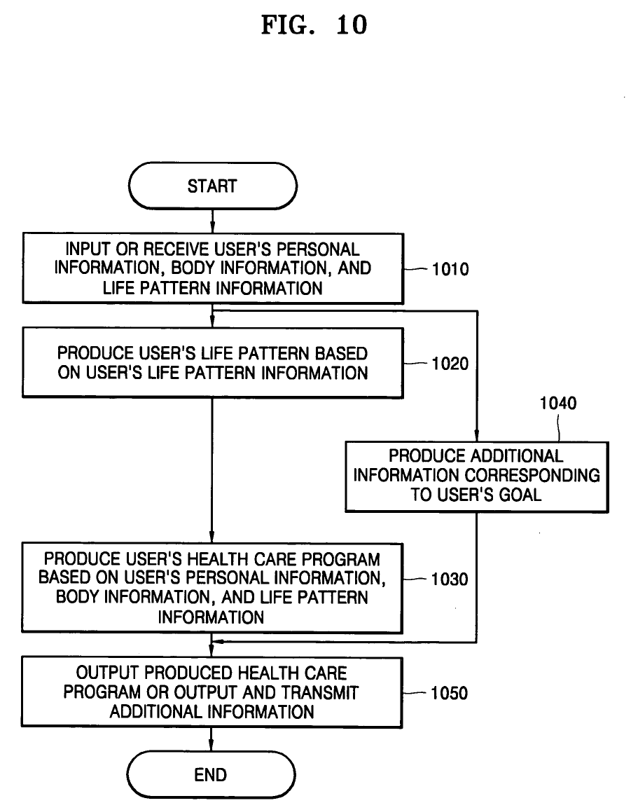

FIG. 10 is a flowchart of a health care method based on a user's life pattern according to an embodiment of the present invention. Referring to FIG. 10, user's personal information, body information, and life pattern information are input by a user or an external device through an input unit or a receiver (Operation 1010). A user's life pattern is produced based on the user's life pattern information (Operation 1020). The life pattern information includes food information including kinds and amounts of user's food intake and sport information including kinds and time of the user's sport. An amount of user's nutritive food intake and an amount of user's consumed nutritive food are calculated from the user's food and sport information. The user's life pattern is produced based on the calculated amount of user's nutritive food intake and amount of user's consumed nutritive food. A user's health care program is produced based on the user's personal information, body information, and life pattern information (Operation 1030). The produced health care program is output to the user or transmitted to a user's external device through an output unit or a transmitter (Operation 1050). Additional information relating to the user's personal information and body information can be produced (Operation 1040). The produced additional information is output to the user or transmitted to the user's external device through the output unit or the transmitter (Operation 1050).

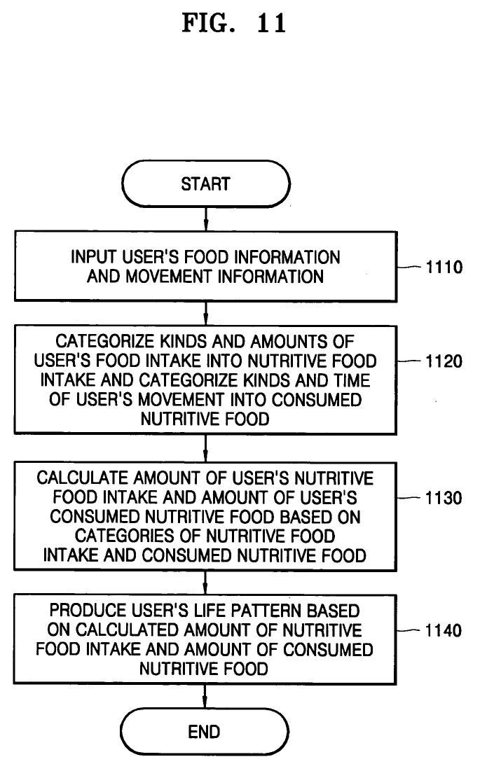

FIG. 11 is a flowchart of Operation 1020 shown in FIG. 10. Referring to FIG. 11, the food information including kinds and amounts of user's food intake and the sport information including kinds and time of the user's sport are input using the input unit or the receiver (Operation 1110). An amount of user's nutritive food intake is mapped to a predetermined category of a user's nutritive food intake table based on the kinds and amounts of user's food intake, and an amount of user's consumed nutritive food is mapped to a predetermined category of a user's consumed nutritive food table based on the kinds and time of the user's sport (Operation 1120). The amount of user's nutritive food intake and the amount of user's consumed nutritive food are calculated based on categories of the mapped user's nutritive food intake and user's consumed nutritive food (Operation 1130). The amount of user's nutritive food intake and the amount of user's consumed nutritive food can be directly calculated by a predetermined device for measuring the amount of user's consumed nutritive food within the scope of the present invention.

The user's life pattern is produced based on the calculated amount of user's nutritive food intake and amount of user's consumed nutritive food (Operation 1140). The produced user's life pattern is based on a predetermined time unit, e.g., an amount of users' nutritive food intake and consumed nutritive food in a day unit.

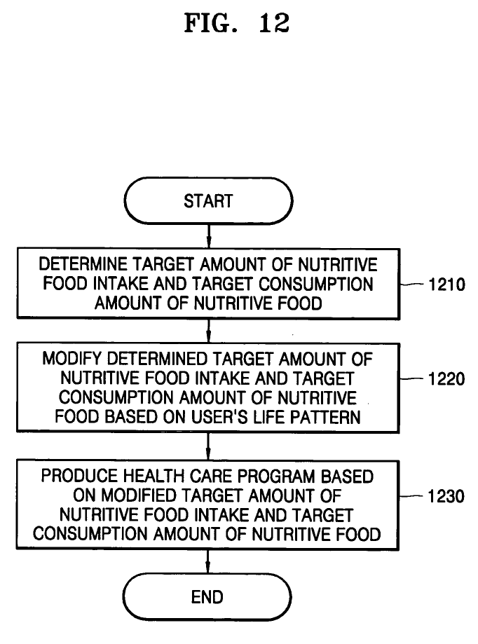

FIG. 12 is a flowchart of Operation 1030 shown in FIG. 10. Referring to FIG. 12, a target amount of nutritive food intake and a target consumption amount of nutritive food are determined based on the user's personal information and body information (Operation 1210). The target amount of nutritive food intake and the target consumption amount of nutritive food are determined by a user's predetermined target obtained by the personal information management agent 210 through the input unit or the receiver. If the user's predetermined target is not established, a standard amount of nutritive food intake and a standard consumption amount of nutritive food are determined based on the user's personal information and body information. The target amount of nutritive food intake and the target consumption amount of nutritive food are modified based on the user's life pattern produced from the life pattern production agent 230 (Operation 1220). When the user has nutritive food in excess of the target amount of nutritive food intake per day, the target amount of nutritive food intake and the target consumption amount of nutritive food are modified. The health care program is produced based on the modified target amount of nutritive food intake and target consumption amount of nutritive food (Operation 1230). To accomplish the modified target amount of nutritive food intake or the modified target consumption amount of nutritive food are modified, food information is provided to the user to have adequate nutritive food, or sport information is provided to the user to consume nutritive food. The provided food information preferably may be information on user's favorite food and the provided sport information preferably may be information on user's favorite sport.

Embodiments of the present invention can also be embodied as computer readable code on a computer readable recording medium. The computer readable recording medium is any data storage device that can store data which can be thereafter read by a computer system. Examples of the computer readable recording medium include read-only memory (ROM), random-access memory (RAM), CD-ROMs, magnetic tapes, floppy disks, optical data storage devices, and carrier waves.

The health care method and apparatus according to the above-described embodiments of the present invention produce a health care program based on a user's life pattern that reflects user's nutritive food intake and user's consumed nutritive food, thereby providing a user with a more accurate and reliable health care program. Also, the produced health care program is based on user's favorite food, user's favorite sport, and user's personal schedule, such that the user can more actively execute the health care program.

The health care method and apparatus according to the above-described embodiments of the present invention provide additional information relating to user's personal and body information, such that the user can more positively execute the health care program.

Although a few embodiments of the present invention have been shown and described, the present invention is not limited to the described embodiments. Instead, it would be appreciated by those skilled in the art that changes may be made to these embodiments without departing from the principles and spirit of the invention, the scope of which is defined by the claims and their equivalents.

What is claimed is:

1. A health care apparatus comprising:
an input unit inputting a user's personal information, body information, and life pattern information;
a calculator producing a health care program based on the input user's personal information, body information, and life pattern information; and
an output unit outputting the produced health care program.

2. The apparatus of claim 1, wherein the calculator comprises:
a life pattern production agent producing a user's life pattern based on the user's life pattern information;
a health care program production agent producing the health care program based on the input user's personal information, body information, and life pattern information; and
a controller controlling information input to and output from each of the agents.

3. The apparatus of claim 2, wherein the calculator further comprises:
a personal information management agent obtaining the user's personal information and storing the obtained personal information; and
a body information management agent obtaining the user's body information and storing the obtained body information.

4. The apparatus of claim 3, wherein each of the agents provides another agent with its information at the request of the another agent.

5. The apparatus of claim 4, wherein the user's personal information comprises a user's name, an ID number, family particulars, an age, a marriage status, a disease history, a goal, a favorite food, or a favorite sport.

6. The apparatus of claim 4, wherein the user's body information comprises a user's weight, height, blood pressure, fat, sugar value, or blood components.

7. The apparatus of claim 4, wherein the user's life pattern information comprises food information on kinds and amount of a user's food intake and sport information on kinds and time of a user's sport.

8. The apparatus of claim 7, wherein the life pattern production agent comprises:
a calculator calculating an amount of user's nutritive food intake and an amount of user's consumed nutritive food based on the user's food intake and the sport information; and
a life pattern producer producing the user's life pattern based on the calculated result.

9. The apparatus of claim 8, wherein an amount of a nutritive food intake corresponding to the kinds and amount of the user's food intake is categorized and an amount of consumed nutritive food corresponding to the kinds and time of the user's sport is categorized, and
wherein the calculator calculates the amount of user's nutritive food intake and the amount of user's consumed nutritive food based on the nutritive food intake category and the consumed nutritive food category.

10. The apparatus of claim 4, wherein the health care program production agent comprises:
a determiner determining a target amount of nutritive food intake and a target consumption amount of nutritive food based on the user's personal information and body information;
a modifier modifying the target amount of nutritive food intake and the target consumption amount of nutritive food based on the user's life pattern; and
a health care program producer producing the health care program based on the modified target amount of nutritive food intake and target consumption amount of nutritive food.

11. The apparatus of claim 10, wherein the determiner determines the target amount of nutritive food intake and the target consumption amount of nutritive food corresponding to the user's predetermined target.

12. The apparatus of claim 10, wherein the determiner determines the target amount of nutritive food intake and the target consumption amount of nutritive food corresponding to standard food information and standard body information.

13. The apparatus of claim 4, further comprising: a memory storing the user's personal information, body information, and life pattern information.

14. The apparatus of claim 4, wherein the calculator further comprises an additional information production agent providing additional information relating to the user's personal information and body information.

15. The apparatus of claim 14, wherein the additional information production agent comprises:
an examination result provider providing results of medical examinations of people having similar information to the user's personal information and body information;
a media information provider providing information on media relating to the user's personal information and body information; and
a community producer searching for a community relating to the user's personal information and body information and providing the user with the searched community, or newly producing the community relating to the user's personal information and body information and providing the user with the produced community.

16. The apparatus of claim 4, wherein the user's personal information, body information, and life pattern information are directly input by the user through the input unit.

17. The apparatus of claim 4, further comprising: a receiver receiving the user's personal information, body information, and life pattern information from an external device.

18. The apparatus of claim 4, further comprising: a transmitter transmitting the health care program to an external device.

19. A health care method comprising:
inputting a user's personal information, body information, and life pattern information;
producing a user's life pattern based on the user's life pattern information;
producing the health care program based on the user's personal information, body information, and life pattern information; and
outputting the produced health care program to the user.

20. The method of claim 19, wherein the user's personal information comprises a user's name, an ID number, family particulars, an age, a marriage status, a disease history, a goal, a favorite food, or a favorite sport.

21. The method of claim 19, wherein the user's body information comprises a user's weight, height, blood pressure, fat, sugar value, or blood components.

22. The method of claim 19, wherein the user's life pattern information comprises food information on kinds and amount of a user's food intake and sport information on kinds and time of a user's sport.

23. The method of claim 22, wherein the producing a user's life pattern comprises:
calculating an amount of user's nutritive food intake and an amount of user's consumed nutritive food based on the user's food intake and the sport information; and
producing the user's life pattern based on the calculated amount of user's nutritive food intake and amount of user's consumed nutritive food.

24. The method of claim 23, wherein an amount of a nutritive food intake corresponding to the kinds and amount of the user's food intake is categorized and an amount of consumed nutritive food corresponding to the kinds and time of the user's sport is categorized, and
wherein the amount of user's nutritive food intake and the amount of user's consumed nutritive food are calculated based on the nutritive food intake category and the consumed nutritive food category.

25. The method of claim 19, wherein the producing the health care program comprises:
determining a target amount of nutritive food intake and a target consumption amount of nutritive food based on the user's personal information and body information;
modifying the target amount of nutritive food intake and the target consumption amount of nutritive food based on the user's life pattern; and
producing the health care program based on the modified target amount of nutritive food intake and target consumption amount of nutritive food.

26. The method of claim 25, wherein the target amount of nutritive food intake and the target consumption amount of nutritive food are determined corresponding to the user's predetermined target.

27. The method of claim 25, wherein the target amount of nutritive food intake and the target consumption amount of nutritive food are determined corresponding to standard food information and standard body information.

28. The method of claim 19, further comprising: providing additional information relating to the user's personal information and body information.

29. The method of claim 28, wherein the additional information comprises results of medical examinations made for people having similar information to the user's personal information and body information, information on media relating to the user's personal information and body information, or information on a community relating to the user's personal information and body information.

30. The method of claim 19, wherein the user's personal information, body information, and life pattern information are directly input by the user.

31. The method of claim 19, wherein the user's personal information, body information, and life pattern information are automatically input from an external device.

32. The method of claim 19, further comprising: transmitting the health care program to an external device.

33. A computer-readable storage medium encoded with processing instructions for causing a processor to execute a health care method, the method comprising:
inputting a user's personal information, body information, and life pattern information;
producing a user's life pattern based on the user's life pattern information;
producing the health care program based on the user's personal information, body information, and life pattern information; and
outputting the produced health care program to the user.
